(19) United States
(12) Patent Application Publication
(10) Pub. No.: US 2020/0345291 A1
(43) Pub. Date: Nov. 5, 2020

(54) Systems and methods for measuring volumes and dimensions of objects and features during swallowing observation

(71) Applicant: Stuart M. Bradley, Apex, NC (US)

(72) Inventor: Stuart M. Bradley, Apex, NC (US)

(21) Appl. No.: 16/400,909

(22) Filed: May 1, 2019

(51) Int. Cl. A61B 5/00; G06T 7/00; G06T 7/62; A61B 1/267; A61B 1/00; A61B 1/05

(52) U.S. Cl. CPC A61B 5/4205; G06T 7/0014; G06T 7/62; G06T 2200/24; A61B 1/0005; A61B 1/05; A61B 1/267

(57) ABSTRACT

Systems and methods for observing and analyzing swallowing are disclosed. According an aspect, a system includes an intubating scope and image capture device configured to capture one or more images of an interior of a subject's throat during swallowing of bolus. The system also includes a computing device configured to receive the captured one or more images. The computing device is also configured to analyze the image(s) to measure an amount of tissue covered by bolus in the one or more images. Further, the computing device is configured to present information indicating the measure of the amount of tissue covered by bolus.

### CROSS REFERENCES TO RELATED APPLICATIONS

This application claims the benefit of priority to U.S. Provisional Application No. 62/665,637, filed May 2, 2018, which is herein incorporated in its entirety.

### COPYRIGHT NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever.

### BACKGROUND

The present invention relates to the field of medical imaging and, more specifically, systems and methods for measuring volumes and dimensions of objects and features during swallowing observation.

Dysphagia is a disorder in which the sufferer has difficulty swallowing. This means that it requires the individual more time and effort to move food or liquid from the mouth to the stomach. Dysphagia may be associated with pain, and in some cases swallowing may be impossible necessitating feeding tubes and/or intravenous hydration and nutrition. There are three phases of swallowing: oral, pharyngeal, and esophageal. A problem may arise during any one of the phases. Problems during the pharyngeal phase include having a difficult time starting to swallow; getting food or liquid into the airway (aspiration); and having some food or liquid remain in the throat after swallowing, referred to as residue.

Medical practitioners, such as speech-language pathologists, can work with individuals having swallowing problems. They can observe an individual's swallowing attempts by the use of an endoscope for viewing an interior of the individual's throat. The endoscope may be configured with a camera for capture of images and video of the interior of the throat to thereby allow the medical practitioner to view the throat interior on a display. Although current endoscopes with cameras are very useful for observing swallowing, there is a desire for improved endoscopy systems for assisting a medical practitioner with measuring objects, such as bolus, and for measuring the size of structures and landmarks observed during a typical swallowing evaluation during an individual's swallowing attempts.

### BRIEF DESCRIPTION OF THE DRAWINGS

Certain illustrative embodiments illustrating organization and method of operation, together with objects and advantages may be best understood by reference to the detailed description follows taken in conjunction with the accompanying drawings in which:

FIG. 1 is a schematic diagram of an example system for use by a medical practitioner to observe and analyze swallowing of an individual or subject in accordance with embodiments of the present disclosure.

FIG. 2 is a flow chart of an example method used to observe and analyze swallowing of an individual or subject in accordance with embodiments of the present disclosure.

FIG. 3 is an image showing the anterior commissure of the true vocal folds (TVCs).

FIGS. 4A and 4B show images that have been captured by a left camera and a right camera, respectively.

FIGS. 4C and 4D illustrate diagrams showing side views of cameras at a determined distance from a plane on which the point is located

FIGS. 5A, 5B, and 5C are front, side, and top views, respectively, of an imaging head illustrating dual cameras consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The following detailed description is made with reference to the figures. Exemplary embodiments are described to illustrate the disclosure, not to limit its scope, which is defined by the claims. Those of ordinary skill in the art will recognize a number of equivalent variations in the description that follows.

The functional units described in this specification have been labeled as computing devices. A computing device may be implemented in programmable hardware devices non-limiting examples of which include processors, digital signal processors, central processing units, field programmable gate arrays, programmable array logic, programmable logic devices, cloud processing systems, graphics processing units, or the like. The computing devices may also be implemented in software for execution by various types of processors. An identified device may comprise executable code and may comprise one or more physical or logical blocks of computer instructions, which may, for instance, be organized as an object, procedure, function, or other construct. Nevertheless, the executable of an identified device need not be physically located together, but may comprise disparate instructions stored in different locations which, when joined logically together, comprise the computing device and achieve the stated purpose of the computing device.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, or materials. In other instances, well-known structure”, materials, or operations are not shown or described in detail to avoid obscuring aspects of the disclosed subject matter.

As referred to herein, the term “user interface” is generally a system by which users interact with a computing device. A user interface can include an input for allowing users to manipulate a computing device, and can include an output for allowing the computing device to present information and/or data or indicate the effects of the user's manipulation. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. As a non-limiting example, a user interface can be a display window or display object, which is selectable by a user of a computing device for interaction. In another non-limiting example, the user can use any other suitable user interface of a computing device, such as a keypad, to select the display icon or display object. As a non-limiting example, the user can use a track ball or arrow keys for moving a cursor to highlight and select the display object.

As used herein, the term “memory” is generally a storage device of a computing device. Examples include, but are not limited to, read-only memory (ROM) and random-access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. As a non-limiting example, exemplary embodiments are intended to cover all software or computer programs capable of enabling processors to implement the disclosed processes. Exemplary embodiments are also intended to cover any and all currently known, related art or later developed non-transitory recording or storage mediums (non-limiting examples of which may include a CD-ROM, DVD-ROM, hard drive, RAM, ROM, floppy disc, or magnetic tape cassette) that record or store such software or computer programs. Exemplary embodiments are further intended to cover such software, computer programs, systems and/or processes provided through any other currently known, related art, or later developed medium (non-limiting examples of which may include transitory mediums, and carrier waves), usable for implementing the exemplary operations disclosed below.

In accordance with the exemplary embodiments, the disclosed computer programs can be executed in many exemplary ways. As non-limiting example, the disclosed computer programs may be executed as an application that is resident in the memory of a device or as a hosted application that is being executed on a server and communicating with the device application or browser via a number of standard protocols, such as TCP/IP, HTTP, XML, SOAP, REST, JSON and other protocols. The disclosed computer programs can be written in exemplary programming languages that execute from memory on the device or from a hosted server. As non-limiting examples, the programming language may be BASIC, COBOL, C, C++, Java, Pascal, scripting languages such as JavaScript, Python, Ruby, PHP, Perl, other programming languages, or combinations thereof.

As referred to herein, the term “computing device” should be broadly construed. It can include any type of computing device, as a non-limiting example, a smart phone, a cell phone, a pager, a personal digital assistant (PDA, e.g., with GPRS NIC), a mobile computer with a smartphone client, or the like. A computing device can also include any type of conventional computer, as a non-limiting example, a desktop computer, a laptop computer, or a tablet computer. A typical mobile device is a wireless data access-enabled device (e.g., an iPHONE® smartphone, a BLACKBERRY® smart phone, a NEXUS ONE™ smart phone, an iPAD™ device, or the like) that is capable of sending and receiving data in a wireless manner using wireless and/or near-field communications protocols. Non-limiting examples of communication protocols may include the Internet Protocol (IP), BLUETOOTH®, WI-FI®, and the wireless application protocol (WAP). This allows users to access information via wireless devices.

Non-limiting examples of wireless devices may include smart phones, mobile phones, pagers, two-way radios, laptop computers, and communicators. Wireless data access is supported by many wireless networks, including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G, 5G and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications networks) on so-called mini- or micro-browsers, which are web browsers with small file sizes that can accommodate the reduced memory constraints of wireless networks.

In a representative embodiment, the mobile device may be a cellular telephone or smart phone that operates over GPRS (General Packet Radio Services), which is a data technology for GSM networks. In addition to a conventional voice communication, a given mobile device can communicate with another such device via many different types of message transfer techniques, including SMS (short message service), enhanced SMS (EMS), multi-media message (MMS), email WAP, paging, or other known or later-developed wireless data formats. Although many of the examples provided herein are implemented on a mobile device, the examples may similarly be implemented on any suitable computing device.

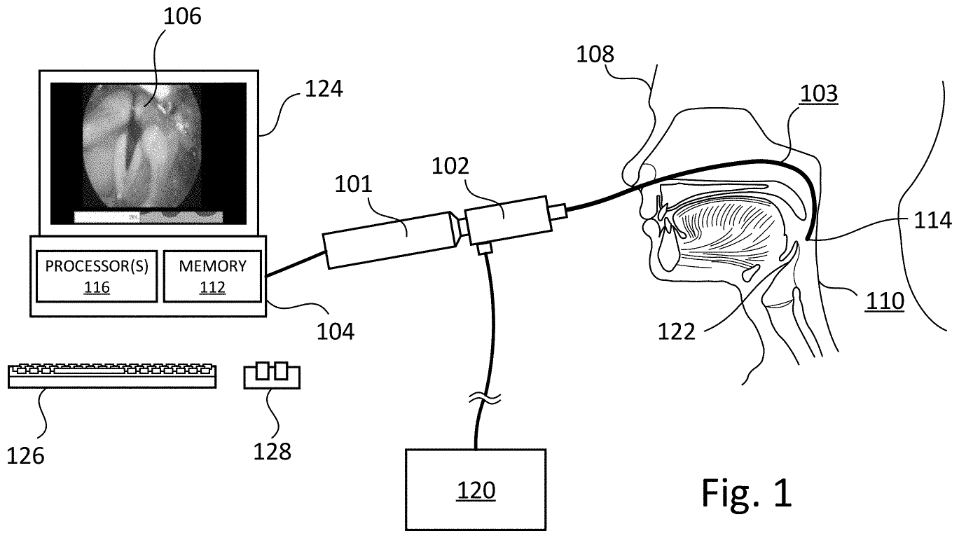

The present disclosure is now described in more detail. As a non-limiting example, FIG. 1 illustrates a schematic diagram of an example system for use by a medical practitioner to observe and analyze swallowing of an individual or subject in accordance with embodiments of the present disclosure. Referring to FIG. 1, the system includes an image capture device 101, an intubating scope 102, an intubating tube 103, and a computing device 104 operably connected to a user interface 106. The system may be used by a medical practitioner, such as a speech-language pathologist, to intubate a throat 110 of an individual 108 for capture of images or video by the image capture device 101. The image capture device 101 may be operably connected to the computing device 104 for receipt of the captured image(s) or video by the computing device 104. The computing device 104 may suitably store the captured image(s) or video in a memory 112. Alternatively, the captured image(s) or video may be stored at a remote location, such as, but not limited to, at a server farm. As depicted in the figure, a distal end 114 of the intubating tube 103 may be suitably positioned by the medical practitioner near the vocal folds 122 of the individual 108 for capture of images or video of this portion of the throat's interior. The computing device 104 may suitably control the image capture device 101 to capture images or video during all or some of the phases of swallowing. As a non-limiting example, images or video may be captured during the pharyngeal phase. The intubating tube 103 may be inserted transnasally into the interior of the subject's throat.

The image capture device 101 may include processing equipment housed in a compartment and that are operably connected to suitable cameras positioned within the distal end 114 of the intubating tube 103. In an example, two cameras are integrated within the distal end 114 of the intubating tube 103 and are each configured to capture one or more images of an interior of the throat 110 of the individual 108. The field-of-view of the cameras may include the epiglottis, the larynx opening into the pharynx, the esophagus, and/or other features in the interior of the throat 110. Therefore, the cameras may capture images and video of the epiglottis, the larynx opening into the pharynx, and the esophagus. The configuration of the image capture device 101 and the intubating scope 102 for capturing images and/or video of the throat's interior will be understood to those of skill in the art.

In another example, the cameras may be positioned at the distal end 114 of the intubating tube 103. In this example, fiber optics may be used to carry light from the throat's interior to the camera, and to deliver light from a light source 120 to the throat's interior for illuminating the interior. In another example, the camera may be suitably affixed to the distal end 114 of the intubating scope 102.

The computing device 104 is configured to receive the captured images. In addition, the computing device 104 can overlay the first image and the second image onto a reference map such that the positioning of the first and second images with respect to each other on the reference map corresponds to the respective fields of view of the first and second cameras. The computing device 104 can also determine, on the reference map, a distance between the feature in the first image and the feature in the second image. The computing device 104 can also determine a measure of another feature of the throat 110 of the individual 108 or an object in the throat 110 of the individual 108 based on a known distance between the first and second cameras, and the determined distance between the feature in the first image and the feature in the second image. The computing device 104 may utilize the differential views of the first camera and the second camera to create a three-dimensional, topographic mapping of the features within the throat 110 of the individual 108 in real time. The created three-dimensional, topographic map may be presented to the user as an image on the display device of the user interface 106 as it is created by the computing device 104. The computing device 104 may include suitable hardware, software, firmware, or combinations thereof for implementing the functionality described herein. As a non-limiting example, the computing device 104 may include one or more processors 116 and the memory 112. The memory 112 may store executable instructions for implementation by the one or more processors 116 for implementing the functionality described herein.

Note that throughout this document references to the two cameras may be made using the terminology of a first camera and a second camera or using the terminology of a left camera and a right camera. The terminology is equivalent and interchangeable. The first camera is the left camera and the second camera is the right camera.

The computing device 104 may comprise one or more devices or components for receiving user input and for presenting information or graphics in accordance with embodiments of the present disclosure. As a non-limiting example, the computing device 104 may comprise a keyboard 126, a mouse 128, a display 124, a printer, or combinations thereof. These components may be operably connected to the computing device 104 via wires or wirelessly as will be understood by those of skill in the art.

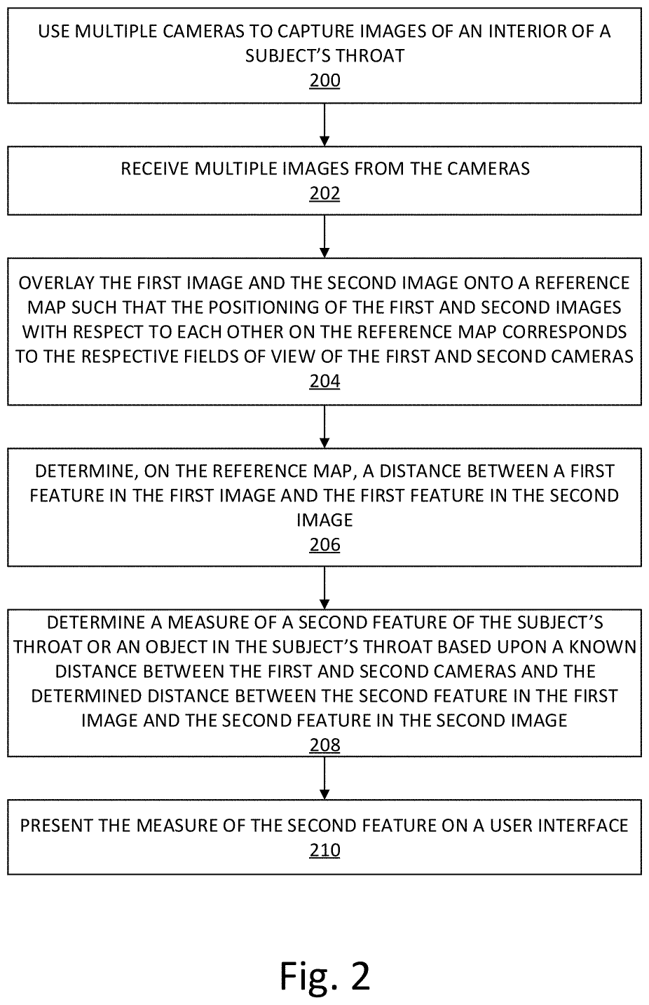

Now turning to FIG. 2, this figure illustrates a flow chart of an example method used to observe and analyze swallowing of an individual or subject in accordance with embodiments of the present disclosure. This example method is described in this example as being implemented by the system shown in FIG. 1, although it should be understood that the method example may alternatively implemented by any other suitable system.

At block 200 the method of FIG. 2 may comprise capturing, by multiple cameras, images of an interior of a subject's throat. As a non-limiting example, the cameras and the intubating scope 102 may be operated to capture images and/or video of the interior of the throat 110 shown in FIG. 1. The images and/or video may be captured prior to or during the pharyngeal phase of swallowing of bolus by the individual 108. It will be understood by skilled medical practitioners that the bolus may include foodstuff and/or liquid prepared for observing the individual's swallowing. Prior to being given to the individual 108 for swallowing, the bolus may be covered at least partially with blue or green food coloring such that the bolus may be easily identified in captured images or video based on the coloring. Techniques for coloring foodstuffs and liquid for identification should be understood by those of skill in the art.

The captured images may include images of the base of the tongue, the posterior pharyngeal wall, and the lateral pharyngeal structures. The computing device 104 may be configured to recognize these and/or other features in the images by use of suitable image recognition techniques. In addition, the captured images may include various objects within the subject's throat such as, but not limited to, bolus and liquids.

The images may be captured by the cameras simultaneously or near simultaneously. As a non-limiting example, capture of an image by one camera may be at the same time or within a fraction of a second of capture of an image by the other camera. This pair of captured images may be overlaid onto a reference map with one another as described in further detail herein.

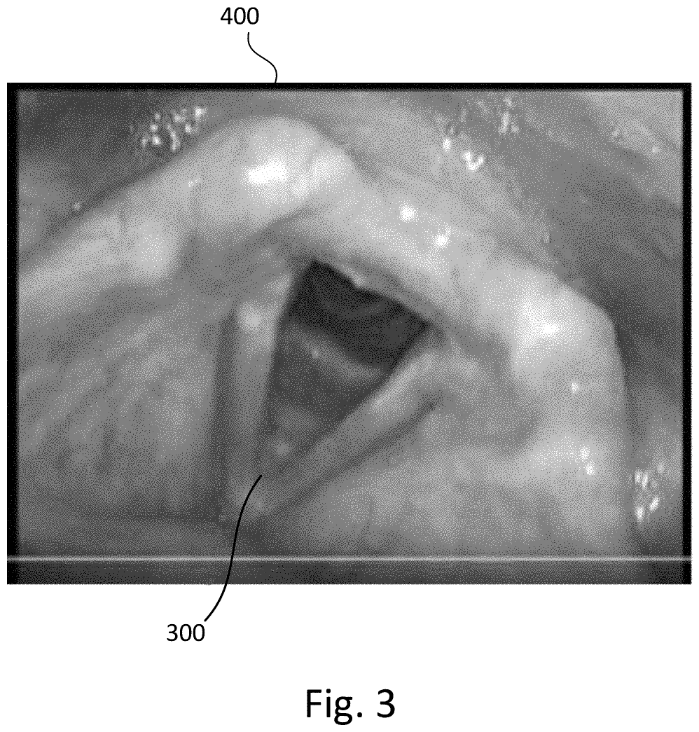

At block 202 the method of FIG. 2 may comprise receiving the captured images from the cameras. Continuing the aforementioned example, the computing device 104 may receive the captured images or video. In this example, the computing device 104 is suitably connected to the cameras for receipt of the captured images or video. The images each include a particular feature of the subject's throat. The computing device 104 may be configured to recognize a first feature via a suitable image recognition technique. In an example, the first feature is the anterior commissure of the true vocal folds of the subject—identified as a reference point 300 in FIG. 3.

At block 204 the method of FIG. 2 may comprise overlaying a first image 402 and a second image 404 onto a reference map such that the positioning of the first image 402 and the second image 404 with respect to each other on the reference map corresponds to the respective fields of view of the first and second cameras. Continuing the aforementioned example, a three-dimensional, topographical reference map, as described in further detail herein below, may be generated by the computing device 104. An image acquired by one of the cameras and an image acquired by the other camera at the same time or nearly at the same time may be overlaid by the computing device onto the reference map. The reference map may correspond to the respective fields of view of the two cameras. As a non-limiting example, the fields of view may be an interior area of the subject's throat.

At block 206 the method of FIG. 2 may comprise determining, on the reference map, a distance between the first feature in the first image and the first feature in the second image. Continuing the aforementioned example, the computing device 104 may determine, on a reference map, a distance between the first feature in the first image and the first feature in the second image. The computing device 104 may be configured to determine coordinates and/or distances on the reference map based on pixels of the first and second images. More particularly, a pixel may have a known size and shape such that coordinates and distances may be determined based on the size and shape and the known arrangement of the pixels with respect to each another.

At block 208 the method of FIG. 2 may comprise determining a measure of a second feature of the subject's throat or an object in the subject's throat based on a known distance between the first and second cameras, and the determined distance between the second feature in the first image and the second feature in the second image. Continuing the aforementioned example, the measure of the second feature of the subject's throat is a length or volume of the second feature of the subject's throat. In another example, the measure of the second feature of the subject's throat is a length or volume of the object in the subject's throat. The determined measure can be a measure of bolus spillage, residual in swallowing, stomach content, mass lesion, or the like.

At block 210 the method of FIG. 2 may comprise presenting, on a user interface, the measure, in terms of any of height, length, width or volume, of the second feature of the subject's throat or the object in the subject's throat. Continuing the aforementioned example, the computing device 104 may control the user interface 106 to present the measure of the second feature of the subject's throat or the object in the subject's throat. As a non-limiting example, the measure may be presented via a display. The measure may be presented as one or more numbers and/or graphics.

In accordance with embodiments, one of the cameras may capture a first sequence of images of the interior of the subject's throat, and another camera may capture a second sequence of images of the interior of the subject's throat. As a non-limiting example, the cameras may each capture video and each frame of video is a unique image of the interior of the subject's throat. Further, the computing device, such as the computing device 104 shown in FIG. 1, may overlay corresponding captured image pairs of the first and second sequence of images onto the reference map such that the positioning of the corresponding images with respect to each other on the reference map corresponds to the respective fields of view of the first and second cameras. Subsequently, the computing device may determine, on the reference map for each captured image pair, a distance between the first feature in each of the images of the pair. The computing device may also determine change in a measure of the second feature of the subject's throat or an object in the subject's throat based on a known distance between the first and second cameras, and the determined distances between the second feature in the images of the pairs.

In accordance with embodiments, a computing device may use captured images as described herein to generate a three-dimensional topographical surface map of the topographical surface area of the subject's pharynx and larynx. The three-dimensional topographical surface map may also be generated by the computing device based on the known distance between the first and second cameras, and the determined distance between the second feature in the first image and the second feature in the second image. As a non-limiting example, the computing device 104 shown in FIG. 1 may implement this functionality.

In an example use case, the presently disclosed subject matter may be used to create motion and still images, and to conduct examinations of pharyngeal/laryngeal swallowing and other medical procedures. Further, the presently disclosed subject matter may provide three-dimensional (3D) visualization of the topology of the individual's pharyngeal/laryngeal structure in real time. In this example, the system for implementing the presently disclosed subject matter can include:

1. A flexible stereoendoscope;

2. A direct computing connection to process and create 3D image from the signal generated by dual cameras placed in the patient's pharynx; and

3. At least one device to view the image in 3D.

The presentation may be simulated 3D on a 2D screen which is achieved by creating simulated depth of field. Alternatively, the presentation may be created using a computer/tv display configured to project 3D imagery that requires specialized viewing glasses for the 3D image to be perceived. Alternatively, the 3D image can be created using specialized goggles that present a stereo image to the user that creates an immersive 3D presentation of the imagery created by the cameras. The computing device may create a three-dimensional topographical map of the surface areas of the internal space known as the pharynx and larynx. The volume of the internal space may be calculated by computing the changes of volume from one frame of imagery to the next. The computing device may also calculate the volume of food bolus, prior to or following swallowing, in the pharynx and larynx based on changes in the calculated volume from one frame to the next.

The presently disclosed subject matter may have various applications. As a non-limiting example, the presently disclosed subject matter may be used to measure volume of bolus spillage and residual in swallowing. In another example, the presently disclosed subject matter may be used to measure stomach content for emptying and stomach function, which may be particularly useful for ileus, PEG (or other) tube feeding. In another example, the presently disclosed subject matter may be used to measure mass lesion size. In another example, the presently disclosed subject matter may be used to measure effect of edema (pre/post inflammatory treatment; effect of foreign body on tissue (i.e., tracheostomy breathing tube)). In another example, the presently disclosed subject matter may be used to measure any other internal body structure or function where it is beneficial to know either the volume of a space/sinus or the size/surface area of a structure.

In accordance with embodiments, a computing device, such as the computing device 104, can determine a measure of a feature of a subject's throat or an object in the subject's throat by initially acquiring images of a reference feature from which further calculations can be made. The captured images can include the base of the tongue, the posterior pharyngeal wall, and the lateral pharyngeal structures (including the complete lateral laryngeal channels which connect the valleculae sulci to the pyriform sulci). In accordance with embodiments, the system may include a stereoscope with two cameras. The cameras can capture two simultaneous, but different, views of the subject's pharynx and laryngeal structures. The views are different because the cameras are offset from each other by a known and fixed distance. This may be done as a continuous video, a series of still photos, and/or a single still photo. Stereovision can be the basis for creating a 3D image.

As mentioned, the known reference feature may be identified prior to calculating distances, volumes, and other measures. In an example, the reference feature is the anterior commissure of the true vocal folds (TVCs). This is the point at the thyroid cartilage where the anterior TVCs attach. The attachment of the bilateral TVCs appear visually as the point of an isosceles triangle. As a non-limiting example, FIG. 3 is a three-dimensional captured image 400 showing the anterior commissure of the TVCs. Referring to FIG. 3, the reference point 300 indicate the point at the thyroid cartilage where the anterior TVCs attach.

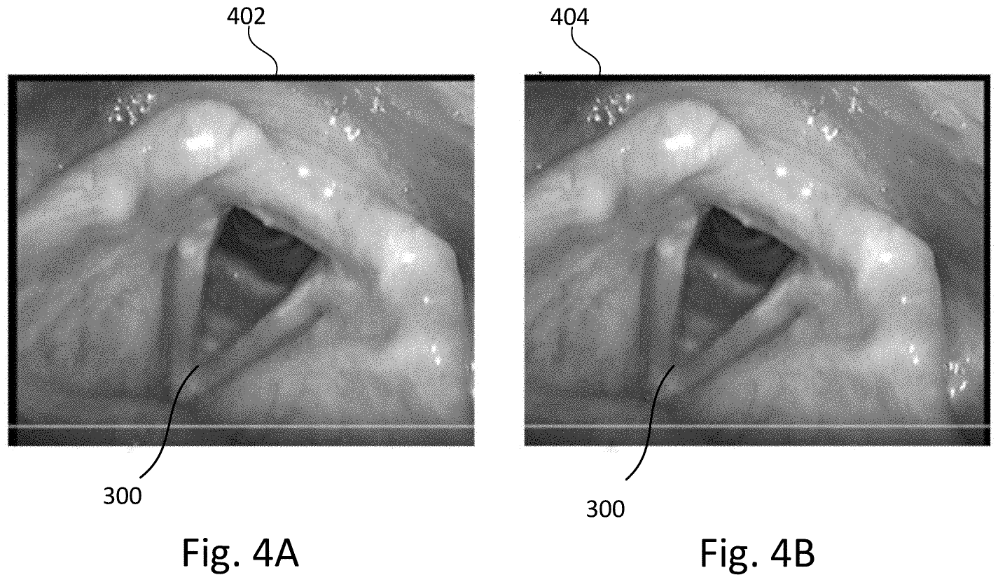

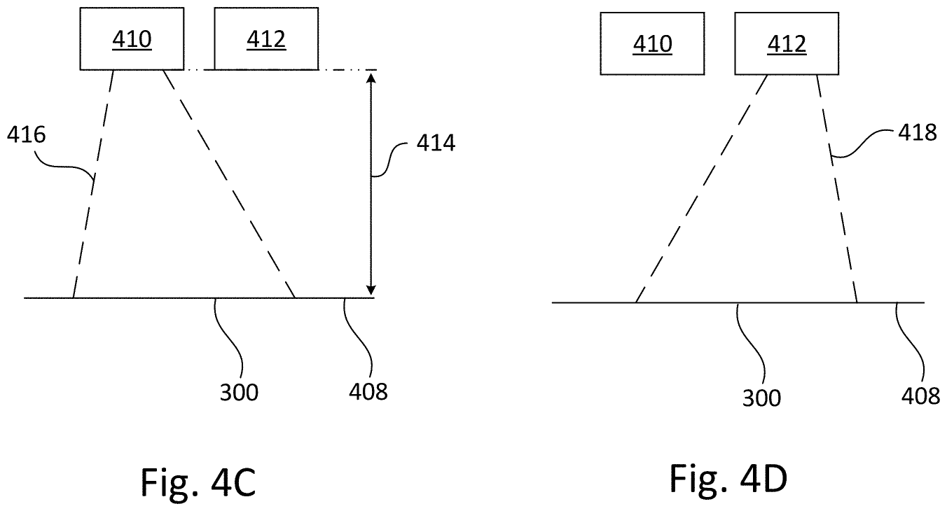

In accordance with embodiments, FIGS. 4A-4D illustrate diagrams depicts techniques and a system for observing and analyzing swallowing of an individual or subject. Referring to FIGS. 4A and 4B, each figure shows a first image 402 and a second image 404 that have been captured by a left camera and a right camera, respectively. The left and right cameras may be placed at the end of an intubating tube. As a non-limiting example, the left and right cameras may be placed at a distal end 114 of an intubating tube 103 shown in FIG. 1 at a known distance in accordance with embodiments of the present disclosure. The images shown in FIGS. 4A and 4B may be captured simultaneously or near simultaneously. The images may be frames of videos captured by the cameras. These images show a reference point 300 at the thyroid cartilage where the anterior TVCs attach.

With continuing reference to FIGS. 4A and 4B, a computing device, such as a computing device 104 shown in FIG. 1, may identify the location of the reference point 300 by use of suitable image recognition software that has been programmed with criteria such that the reference point 300 may be identified by pixel location, such as the coordinates in a pixel array for each image. As a non-limiting example, in the left image, the reference point 300 may be located 738 pixels from the left side of the image and 60 pixels from the bottom of the image. Further, as a non-limiting example, in the right image, the reference point 300 may be located 732 pixels from the left side of the image and 60 pixels from the bottom of the image. The offset in pixels—6 pixels—between the images is due to the spacing between the cameras and their distance to the thyroid cartilage where the anterior TVCs attach. Accordingly, the x,y location of the reference point 300 or target in the first image 402 and in the second image 404 is known.

FIGS. 4C and 4D illustrate diagrams showing side views of a left camera 410 and a right camera 412 at a correct camera distance 414 from a plane 408 on which the reference point 300 is located. Because the left camera 410 and the right camera 412 may have different positions, alignment angles, or both, the left camera 410 may have a left camera field of view 416, the right camera 412 may have a right camera field of view 418, and the left camera field of view 416 may be different from the right camera field of view 418 resulting in captured images that are similar but offset in one or more directions.

Because the geometry, dimensions, and placement of the left camera 410 and the right camera 412 within the distal end 114 of the intubating tube 103 are known by design, the offset in pixels for the reference point 300 determined by comparing the two images may serve as verification that the distal end 114 of the intubating tube 103 is positioned at the correct camera distance 414 from the reference point 300 in a throat 110. The computed pixel distance may be correct for a specific camera distance between the distal end 114 of the intubating tube 103 and the reference point 300 on the throat 110. If the intubating tube 103 is inserted too far into the throat 110 of an individual 108, then the distal end 114 of the intubating tube 103 may be closer to the reference point 300 than the correct camera distance 414 and the pixel distance determined by comparing the images may increase. If the intubating tube 103 insufficiently inserted, then the distal end 114 of the intubating tube 103 may be farther away from the reference point 300 than the correct camera distance 414 and the pixel distance determined by comparing the images may decrease.

In some embodiments, the computing device 104 may provide an on-screen reticle or graticule as part of a user interface 106 and the on-screen reticle or graticule may change visually to indicate that the distal end 114 of the intubating tube 103 is at the correct camera distance 414. As a non-limiting example, the on-screen reticle or graticule may turn green when the distal end 114 of the intubating tube 103 is at the correct camera distance 414.

In some embodiments, the user may be able to specify a value to use for the correct camera distance 414 and the computing device 104 may adjust all calculations based upon the correct camera distance 414 that the user has selected.

The computing device may receive the first image 402 and the second image 404 in accordance with embodiments of the present disclosure. Subsequently, the computing device 104 may overlay the first image 402 and the second image 404 onto a reference map such that the positioning of the first image 402 and the second image 404 with respect to each other on the reference map corresponds to respective fields of view of the cameras. The distance between the left camera 410 and the right camera 412 is known to be the correct camera distance 414 as described herein for use in determining a measure of a feature of the subject's throat or an object in the subject's throat. This determination may be made based on the correct camera distance 414 and the determined number of pixels separating the reference point 300 in the overlaid images—the first image 402 and the second image 404, shown in FIGS. 4A and 4B, respectively. The computing device may use this information to generate the measurements. In addition, the computing device may use the information to generate a three-dimensional topographic surface map of a surface area of the subject's pharynx and larynx based on the first image 402 and the second image 404, the known distance between the left camera 410 and the right camera 412, and the determined distance between the reference point 300 in the overlaid images. In accordance with embodiments, to calculate measurements, a computing device may create an overlay onto a combination of images for the user to guide the computing device towards creating a known reference point. This can provide the computing device with a target for which the computing device can use to create a first measurement on which subsequent measurements can be based and determined. By using the known pixel offsets from comparing the images from each sourced camera in comparison to the overlaid target created by the computing device and the user, the computing device can now create a scaled measurement of structures and bolus inside the pharynx. In an example of implementing this functionality, the computing device can map out the distance between two known points created by the overlay offset with one point coming from each image. The computing device may use these to complete the mathematical application of the Pythagorean Formula that a\hat{ }2+b\hat{ }2=c\hat{ }2, where a is from one camera's image and b is from the other camera's image, and c can be the height of the scope tip from the target point that was set by the user and computer. Knowing the height from the image can allow the user to return to the known target point repeatedly and thereby create comparison measurements from one image to the next whether done as a series of images generated by video camera or single still images. The computing device can now also use that calculated measurement to establish scale, calculated volume, and measurements from various points that are being visualized. Using the measurement, the computing device can now also draw an accurate topographic 3D map of the internal “landscape” that is within its field of view. The topographic 3D map may permit the measurement and calculation of the size of lesions and also measure the volume of any food or liquid bolus remaining in the pharynx or larynx to assist in the diagnosis of swallow disorders, also known as dysphagia. The analysis of the measurement data may also permit the system to provide insights regarding edema, such as edema caused by intubation during surgery. There is a high rate of re-intubation in older patients, and edema measurements may permit the analysis of whether edema serious enough to cause re-intubation was a result of the initial intubation in support of surgery. Additional measurements may be taken after radiology treatment to measure the efficacy of the treatment. Measuring the volume, surface area, and location of lesions and/or cancerous masses before and after treatment may produce information about the shrinkage of the masses or lesions, enabling a more accurate gauge of whether the radiology treatment is working and how well. Measurement data from the system may be accumulated to create normative values for the size and volume of pharynx and larynx features for patients with given known values. As a non-limiting example, an adult male six feet tall, weighing 220 pounds would be expected to have a certain set of size and volume measurements. This normative data may then be compared against measured data from a previously unmeasured patient to provide information to a physician in support of a diagnosis.

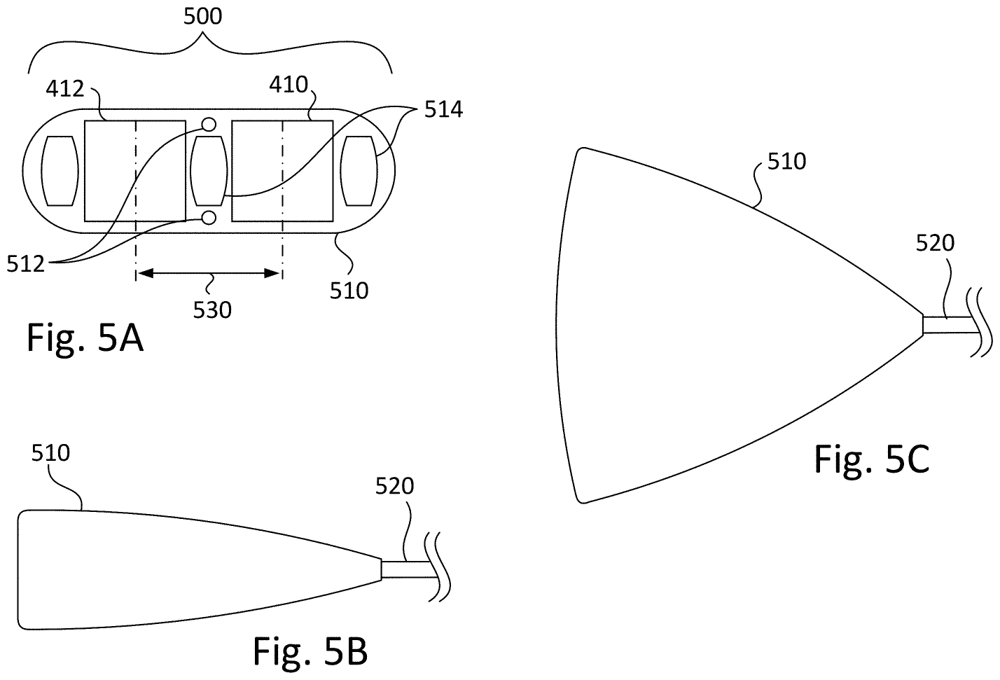

Turning now to FIGS. 5A, 5B, and 5C, a distal end 114 of an intubating tube 103 consistent with an embodiment is shown in a front view (5A), a side view (5B), and a top view (5C). An imaging head 500 may comprise instrumentation contained within a housing 510 and may couple to an intubating scope 102 via an instrumentation cable 520.

The front (5A) of the imaging head 500 may comprise a left camera 410, a right camera 412, and one or more sources of illumination 514. Articulation endpoints 512 of the instrumentation cable 520 may also terminate at the front of the imaging head 500. The articulation endpoints 512 may be the ends of articulation cables in the instrumentation cable 520 that allow the imaging head 500 to be steered. The one or more sources of illumination 514 may provide illumination for imaging purposes. In some embodiments, the one or more sources of illumination 514 may be one or more white LED's. In some embodiments, the one or more sources of illumination 514 may be the ends of optical light pipes that originate at the intubating scope 102 and terminate at the imaging head 500.

The left camera 410 and the right camera 412 may be separated by an inter-camera spacing 530 which is known and which is used in distance computations by a computing device 104. As a non-limiting example, the left camera 410 and the right camera 412 may be charge-coupled devices (CCDs). In some embodiments, the left camera 410 and the right camera 412 may each comprise an array of 400 by 400 pixels.

The instrumentation cable 520 may comprise electrical cabling, optical cabling, mechanical cabling, or combinations thereof. As non-limiting examples, the instrumentation cable 520 may provide electrical power for the left camera 410, the right camera 412, and the one or more sources of illumination 514, may support and steer the imaging head 500, and may transfer images from the left camera 410 and the right camera 412.

In some embodiments, the left camera 410 and the right camera 412 may reside within the intubating scope 102 and images may be sent from the distal end 114 of the intubating tube 103 to the cameras via optical cables located within the instrumentation cable 520.

It is noted that the computing device implemented the functionality described herein may be communicating with any suitable computing device such as, but not limited to, a smartphone, router, laptop computer, desktop computer, or the like.

The present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, as a non-limiting example, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, as a non-limiting example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (as a non-limiting example, through the Internet using an Internet Service Provider). In some embodiments, electronic circuitry including, as a non-limiting example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. As a non-limiting example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While certain illustrative embodiments have been described, it is evident that many alternatives, modifications, permutations and variations will become apparent to those skilled in the art in light of the foregoing description.

What is claimed is:

1. A system comprising:
an intubating scope and image capture device comprising a first camera and a second camera that are each configured to capture one or more images of an interior of a subject's throat; and
a computing device including at least one processor and memory configured to:
receive, from the first camera, a first image including a reference feature of the subject's throat;
receive, from the second camera, a second image including said reference feature of the subject's throat;
overlay the first image and the second image onto a reference map such that the positioning of the first and second images with respect to each other on the reference map corresponds to the respective fields of view of the first and second cameras;
determine, on the reference map, a distance between the reference feature in the first image and said reference feature in the second image; and
determine a measure of other features of the subject's throat or an object in the subject's throat based on a known distance between the first and second cameras, and the determined distance between the other features in the first image and the other features in the second image;
create a three-dimensional topographic map of all measured features and present said three-dimensional topographic map to a user.

2. The system of claim 1, where the computing device is configured to identify said reference feature as a known feature of the subject's throat.

3. The system of claim 1, where the measure of the other features of the subject's throat are the length or volume of the other features of the subject's throat or of an object in the subject's throat.

4. The system of claim 1, where the computing device is configured to determine a distance between the first and second cameras and the reference feature based on the determined distance and the known distance between the first and second cameras.

5. The system of claim 1, where the computing device is configured to determine coordinates and distances on the reference map based on pixels of the first and second images.

6. The system of claim 1, where the first camera is configured to capture a first sequence of images of the interior of the subject's throat, where the second camera is configured to capture a second sequence of images of the interior of the subject's throat, and where the computing device is configured to:
overlay corresponding captured image pairs of the first and second sequence of images onto the reference map such that the positioning of the corresponding images with respect to each other on the reference map corresponds to the respective fields of view of the first and second cameras;
determine, on the reference map for each captured image pair, a distance between the reference feature in the images of the pair; and
determine change in a measure of another feature of the subject's throat or an object in the subject's throat based on a known distance between the first and second cameras, and the determined distances between the reference feature in the images of the pairs.

7. The system of claim 1, where the computing device is configured to generate a three-dimensional topographic surface map of a surface area of the subject's pharynx and larynx based on the first and second images, the known distance between the first and second cameras, and the determined distance between the reference feature in the first image and said reference feature in the second image.

8. The system of claim 1, where the computing device comprises a user interface, and where the computing device is configured to control the user interface to present the three-dimensional topographic measure of each of the features of the subject's throat or an object in the subject's throat.

9. The system of claim 1, where the first and second images are captured simultaneously or substantially simultaneously.

10. A method comprising:
using a first camera and a second camera to capture one or more images of an interior of a subject's throat;
receiving, from the first camera, a first image including a reference feature of the subject's throat;
receiving, from the second camera, a second image including said reference feature of the subject's throat;
overlaying the first image and the second image onto a reference map such that the positioning of the first and second images with respect to each other on the reference map corresponds to the respective fields of view of the first and second cameras;
determining, on the reference map, a distance between the reference feature in the first image and said reference feature in the second image;
determining a measure of other features of the subject's throat or an object in the subject's throat based on a known distance between the first and second cameras, and the determined distance between the reference feature in the first image and the feature in the second image;
presenting the measure of each feature in a subject's throat on a display accessible to a user.

11. The method of claim 10, further comprising identifying the reference feature as a known feature of the subject's throat.

12. The method of claim 10, where the measure of each of the other features of the subject's throat comprises a length or volume of each of the other features of the subject's throat or of an object in the subject's throat.

13. The method of claim 10, further comprising determining a distance between the first and second cameras and the reference feature based on the determined distance and the known distance between the first and second cameras.

14. The method of claim 10, further comprising determining coordinates and distances on the reference map based on pixels of the first and second images.

15. The method of claim 10, further comprising:
using the first camera to capture a first sequence of images of the interior of the subject's throat;
using the second camera is configured to capture a second sequence of images of the interior of the subject's throat;
overlaying corresponding captured image pairs of the first and second sequence of images onto the reference map such that the positioning of the corresponding images with respect to each other on the reference map corresponds to the respective fields of view of the first and second cameras;
determining, on the reference map for each captured image pair, a distance between the feature in the images of the pair; and
determining change in a measure of another feature of the subject's throat or an object in the subject's throat based on a known distance between the first and second cameras, and the determined distances between the reference feature in the images of the pairs.

16. The method of claim 10, further comprising generating a three-dimensional topographic surface map of the surface area of the subject's pharynx and larynx based on the first and second images, the known distance between the first and second cameras, and the determined distance between the reference feature in the first image and the feature in the second image.

17. The method of claim 10, further comprising using a user interface to present the three-dimensional topographic map of each of the other features of the subject's throat or an object in the subject's throat.

18. The method of claim 10, where the first and second images are captured simultaneously or substantially simultaneously.
